(19) United States
(12) Patent Application Publication
(10) Pub. No.: US 2015/0050678 A1
(43) Pub. Date: Feb. 19, 2015

(54) MODULAR ANALYTICAL TEST METER

(71) Applicant: LifeScan Scotland Limited, Inverness-shire (GB)

(72) Inventors: David Elder, Inverness (GB); Allan Faulkner, Inverness (GB); Allan Macrae, Inverness (GB); Keith Lawrie, Inverness (GB)

(73) Assignee: LifeScan Scotland Limited, Inverness-shire (GB)

(21) Appl. No.: 13/965,827

(22) Filed: Aug. 13, 2013

(51) Int. Cl. C12Q 1/54

(52) U.S. Cl. CPC C12Q 1/54
USPC 435/14; 435/287.1

(57) ABSTRACT

A modular analytical test meter includes a meter chassis or body and a plug-in analytical module that is electrically and mechanically attached in a releasable fashion to the meter chassis. When attached, the plug-in analytical module includes resident circuitry configured to measure for an analyte of interest from an analytical test strip, the module further including stored coded information such as firmware updates that can be utilized by the existing test meter without requiring replacement of an entire system.

### TECHNICAL FIELD

The application relates generally to the field of analytical test systems and more specifically to a portable analytical test meter, such as used for measuring blood glucose, and which is modular in design.

### BACKGROUND

Analyte detection in physiological fluids, e.g., blood or blood-derived products, is of ever increasing importance in today's society. Analyte detection assays find use in a variety of applications, including clinical laboratory testing, home testing, etc., where the results of such testing play a prominent role in diagnosis and management in a variety of disease conditions. Analytes of interest include glucose in diabetes management, cholesterol, and the like. In response to this growing importance of analyte detection, a variety of analyte detection protocols and devices have been developed for both clinical and home use.

One type of system that allows people to conveniently monitor their blood glucose levels includes a sensor (e.g., a disposable test strip) for receiving a blood sample from a user, and a meter that “reads” the test strip to determine the glucose level in the blood sample. The test strip typically includes electrical contacts for mating with the meter and a sample chamber that contains reagents (e.g., glucose oxidase and a mediator) and electrodes. To begin the test, the test strip is inserted into the meter and the user applies a blood sample to the sample chamber. The meter then applies a voltage to the electrodes to cause a redox reaction and the meter measures the resulting current and calculates the glucose level based on the current. After the test is completed, the test strip can be disposed.

It should be emphasized that frequent measurements of blood glucose levels may be critical to the long-term health of many users. As a result, there is a need for blood glucose measuring systems that are easy to use. One example of a known portable typical analytical meter used for measuring blood glucose is depicted in FIG. 1. The test meter 10 is defined by a single unitary housing 11 that retains a plurality of components. A strip port opening 12 extends within the housing 11 to a strip port connector circuit. The strip port connector circuit includes a plurality of contacts that engage the electrodes 13 of an inserted test strip 14, for example, for measuring blood glucose such as in home test kits. A person (not shown) uses a lancelet or other means (not shown) to obtain a small sample of blood that is dispensed onto a portion of the test strip 14. An electrochemical cell 16 provided as part of the test strip 14 is electrically connected to the strip electrodes 13 that are electrically engaged by the test meter 10 through the contacts in the strip port connector circuit. The analyte (blood glucose) concentration can then be measured and displayed on a display screen 15.

If the strip port connector of test meters, such as those depicted in FIG. 1, becomes damaged or contaminated through the repetitive use of test strips, this usually requires replacement of the entire test meter. Replacement of an entire test meter is significant in terms of cost.

In addition, the design of test strips is known to change over time based on size and electrode configurations. A typical analytical test meter is not configured for updated test strip versions, meaning that the meter would be potentially obsolete and require replacement based upon the introduction of new strip platforms.

Still further and while the processing logic of many analytical test meters can be updated, such as via a USB or other form of connection, it would be advantageous to transport new code and instructions to the user and ensure that the new code has not been corrupted. Other known designs revolve about the transmission of communication bidirectionally via a web server or in some instances using a smart phone application. Each of these latter techniques, however, requires the user to have the correct equipment to download and upgrade the test meter.

Still further, a specific production line is required for the manufacture of each type of test meter. For example, if three (3) different meter capabilities were required, (e.g., low, medium and premium) and each of these capabilities had the additional option of three (3) different test strip types, this would require a total of nine (9) meter models, and consequently a corresponding number of disparate and custom production lines.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 is a perspective view of a prior art analytical test meter;

FIGS. 2A-2B are perspective views of an analytical module and meter chassis in accordance with an exemplary embodiment;

FIG. 2C illustrates, in schematic form, electronic components contained within the meter chassis depicted in FIG. 2B in accordance with an exemplary embodiment;

FIG. 3 illustrates, in schematic form, additional details of the electronic components of the analytical module of FIG. 2A in accordance with an exemplary embodiment;

FIG. 4 illustrates, in schematic form, components of the analytical module of FIG. 2A in accordance with an exemplary embodiment;

FIG. 5 illustrates a perspective view of an analytical module and meter chassis in accordance with another exemplary embodiment; and

FIG. 6 illustrates a flow chart of an exemplary method of operating the test meter of FIG. 2B.

### MODES OF CARRYING OUT THE INVENTION

The following description relates to a modular analytical test meter in accordance with certain exemplary embodiments and should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Throughout the course of discussion and in order to provide a suitable frame of reference with regard to the accompanying drawings, certain terms are often used such as “upper”, “lower”, “proximal”, “distal” , “top”, “bottom” and the like. These terms are not intended, unless specifically indicated, to affect the overall scope of the present invention.

As used herein, the terms “patient” or “user” refer to any human or animal subject and are not intended to limit the systems or methods to human use, although use of the subject invention in a human patient represents a preferred embodiment.

The term “sample”, as used herein, means a volume of a liquid, solution or suspension, intended to be subjected to qualitative or quantitative determination of any of its properties, such as the presence or absence of a component, the concentration of a component, e.g., an analyte, etc. The embodiments of the present invention are applicable to human and animal samples of whole blood. Typical samples in the context of the present invention as described herein include blood, plasma, red blood cells, serum and suspensions thereof.

The term “about” as used in connection with a numerical value throughout the description and claims denotes an interval of accuracy, familiar and acceptable to a person skilled in the art. The interval governing this term is preferably ±10%. Unless specified, the terms described above are not intended to narrow the scope of the invention as described herein and according to the claims.

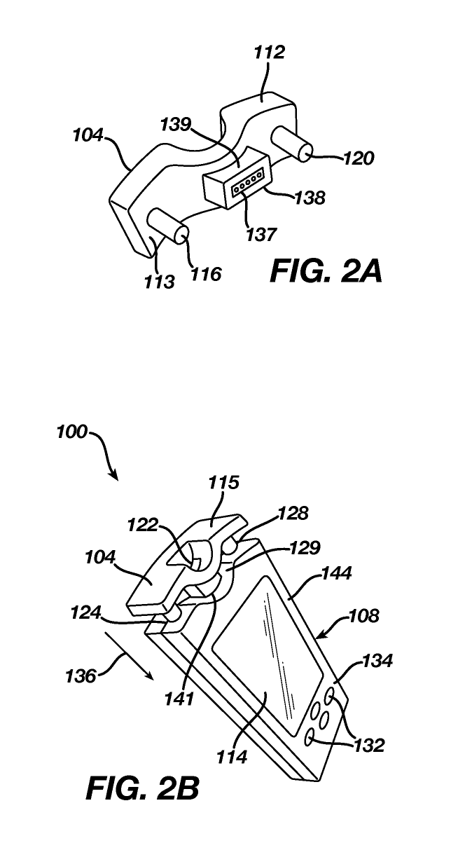

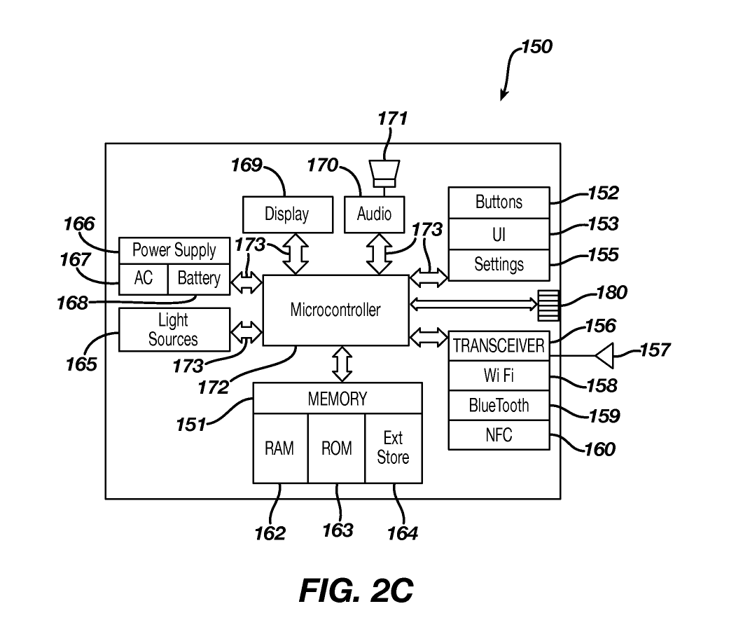

Referring to FIGS. 2A and 2B, there is shown an analytical test meter 100 made in accordance with an exemplary embodiment. The depicted test meter 100 includes a plug-in analytical module 104 that can be releasably engaged with a meter chassis 108. As discussed herein, the plug-in analytical module 104 is provided to receive an analytical test strip 14 for determining the concentration of an analyte of interest from a bodily fluid sample deposited onto the test strip 14. The analytical module is designed to both mechanically and electrically engage the meter chassis 108 and to cooperate therewith. The plug-in analytical module 104 is defined by a module housing 112 having a distal end 113 and an opposing proximal end 115. A pair of spaced engagement pins 116, 120 extend outwardly from the distal end 113 and are appropriately sized to mechanically engage the meter chassis 108, defined by a chassis housing 144, the latter having spaced openings 124, 128 at a proximal end 129 of the chassis housing 144 sized for receiving the pins 116, 120 when the module 104 is engaged or inserted according to arrow 136 with the proximal end 129 of the chassis housing 144. A strip port opening 122 is formed in the proximal end 115 of the analytical module 104, the strip port opening 122 allowing access to a strip port connector (“SPC”) 139 within enclosure 138 having a set of electrical contacts (not shown) in the form of prongs or other members suitable for providing electrical contact with an inserted test strip 14. The enclosure 138 extends from the distal end 113 of the analytical module housing 112 between the spaced engagement pins 116, 120, wherein the enclosure 138 is fittable within a suitably sized recess 141 that is defined in the proximal end 129 of the chassis housing 144. The enclosure 138 also includes a number of electrical contacts 137, electrically connected to the SPC 139, which form an electrical module interface, and includes a power interface, that connects with a mating electrical chassis interface 180 (FIG. 2C) positioned within the recess 141. The electrical interfaces 137, 180 may include male/female type ohmic electrical connectors using pins or other suitable connectors. Taken together, the enclosure 138, engagement pins 116, 120, electrical contacts 137, and distal end 113 form an electrical and mechanical module interface. A corresponding electrical and mechanical chassis interface for engaging therewith is formed by openings 124, 128, recess 141, the chassis electrical interface 180, and the proximal end 129 of the chassis housing 144.

According to this specific embodiment, the meter chassis 108 and the plug-in analytical module 104 each include features that provide mechanical, as well as electrical interconnection in a releasable fashion. Mechanical keying, such as using the analytical module's engagement pins 116, 120 appropriately sized and spaced to fit in spaced openings 124, 128 of an authorized version of chassis housing 144 is provided in order to assure that incompatible analytical modules cannot be connected onto an unauthorized meter chassis. Authorized versions are those meter chassis 108 that are designed to be compatible with a particular analytical module 104. For example, a meter chassis 108 having a segmented LCD display may not be compatible with an analytical module 104 that requires a color display to operate its user interface. It should be noted in passing that the above-noted features of the herein described mechanical interface can be reversed. For example, a set of engagement pins can be provided on the proximal end 129 of the chassis housing 144 with a complementary set of spaced openings being provided on the distal end 113 of the plug-in analytical module 104 to provide a suitable connection between the chassis 108 and the plug-in analytical module 104. A person of ordinary skill in the art will recognize that various mechanical features may be designed so that the analytical module 104 may be selectively engaged with a meter chassis 108. Such designs are considered to be within the scope of the embodiments disclosed herein.

Still referring to FIGS. 2A and 2B and as previously noted, the meter chassis 108 is defined by a chassis housing 144 that retains a number of components, as described below with reference to FIG. 2C. A user interface includes a plurality of interface buttons 132 for powering on and off and also for operating the test meter 100, the interface buttons according to this version being provided on a top surface 134 of the chassis housing 144. In addition, a display 114 such as an LCD display is further provided on the top surface 134 of the meter chassis 108. Each of the meter chassis 108 and analytical plug-in module 104 according to the exemplary embodiment can be manufactured from a durable plastic or other suitable structural material.

FIG. 2C illustrates components of the analytical test meter 100 in simplified schematic form as disposed within the housing 144 of the meter chassis 108 for purposes of this embodiment. The electronic components of the analytical test meter 100 can be disposed on, for example, a printed circuit board situated within the meter housing 144 and forming the data management unit (DMU) 150 of the test meter 100. The plurality of user interface buttons 132 communicate with the DMU 150 via the user interface module 152 and can be configured to allow the entry of data, to prompt an output of data, to navigate menus presented on the display 114, and to initiate execution of commands by the microcontroller 172. Output data can include values representative of analyte concentration presented on the display 114 under control of microcontroller 172 via display driver 169. Input information may be requested via prompts presented on the display 114 and can be stored in the memory module 151 of the analytical test meter 100. Although the buttons 132 are shown herein as separate switches, a touch screen interface on display 114 with virtual buttons may also be utilized.

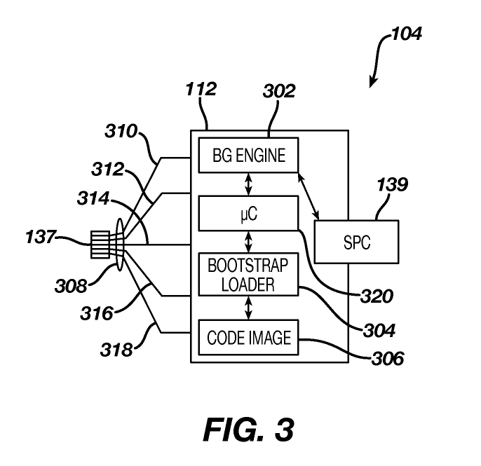

The DMU 150 includes a processing unit 172 in the form of a microprocessor, a microcontroller, an application specific integrated circuit (“ASIC”), a mixed signal processor (“MSP”), a field programmable gate array (“FPGA”), or a combination thereof, and is electrically connected to various electronic modules included on, or connected to, the printed circuit board, as well being connected to the analytical module 104 via chassis electrical interface 180. The processing unit 172 is electrically connected to, for example, the SPC 139 and a bodily sample analyte engine, such as a blood glucose engine 302 (FIG. 3), in the analytical module 104 via the chassis electrical interface 180 coupled to the module electrical interface 137. The analytical module 104 is thus electrically connected to the meter chassis 108 during sample analyte testing, such as blood glucose testing.

A display module 169, which may include a display processor and display buffer, is electrically connected to the processing unit 172 over the electrical interface 173 for receiving and displaying output data, and for displaying user interface input options under control of processing unit 172. The structure of the user interface, such as menu options, is stored in user interface module 153 and is accessible by processing unit 172 for presenting menu options to a user of the analytical test meter 100. An audio module 170 includes a speaker 171 for outputting audio data received or stored by the DMU 150. Audio outputs can include, for example, notifications, reminders, and alarms, or may include audio data to be replayed in conjunction with display data presented on the display 114. Such stored audio data can be accessed by the processing unit 172 and executed as playback data at appropriate times. A volume of the audio output is controlled by the processing unit 172, and the volume setting can be stored in settings module 155, as determined by the processor or as adjusted by the user. The processing unit 172 may have electrical access to a digital time-of-day clock connected to the printed circuit board for recording dates and times of blood glucose or other sample analyte measurements in memory module 151, which may then be accessed, uploaded, or displayed at a later time as necessary.

The display 114 can alternatively include a backlight whose brightness may be controlled by the processing unit 172 via a light source control module 165. Similarly, the user interface buttons 132 may also be illuminated using LED light sources electrically connected to processing unit 172 for controlling a light output of the buttons. The light source module 165 is electrically connected to the display backlight and processing unit 172. Default brightness settings of all light sources, as well as settings adjusted by the user, are stored in a settings module 155, which is accessible and adjustable by the processing unit 172.

A memory module 151, that includes, but is not limited to, volatile random access memory (“RAM”) 162, a non-volatile memory 163, which may comprise read only memory (“ROM”) or flash memory, and a circuit 164 for connecting to an external portable memory device, for example, via a USB data port, is electrically connected to the processing unit 172 over a electrical interface 173. External memory devices may include flash memory devices housed in thumb drives, portable hard disk drives, data cards, or any other form of electronic storage devices. The on-board memory can include various embedded applications and stored algorithms in the form of programs executed by the processing unit 172 for operation of the analytical test meter 100, as will be explained below. On board memory can also be used to store a history of a user's sample analyte measurements, such as blood glucose measurements, including dates and times associated therewith. Using the wireless transmission capability of the analytical test meter 100, as described below, such measurement data can be transferred via wired or wireless transmission to connected computers or other processing devices.

A wireless module 156 may include transceiver circuits for wireless digital data transmission and reception via one or more internal digital antennas 157, and is electrically connected to the processing unit 172 over electrical interface 173. The wireless transceiver circuits may be in the form of integrated circuit chips, chipsets, programmable functions operable via processing unit 172, or a combination thereof. Each of the wireless transceiver circuits is compatible with a different wireless transmission standard. For example, a wireless transceiver circuit 156 may be compatible with the Wireless Local Area Network IEEE 802.11 standard known as WiFi. Transceiver circuit 158 may be configured to detect a WiFi access point in proximity to the analytical test meter 100 and to transmit and receive data from such a detected WiFi access point. A wireless transceiver circuit 159 may be compatible with the Bluetooth protocol and is configured to detect and process data transmitted from a Bluetooth beacon in proximity to the analytical test meter 100. A wireless transceiver circuit 160 may be compatible with the near field communication (“NFC”) standard and is configured to establish radio communication with, for example, another NFC compliant device in proximity to the analytical test meter 100 to initiate a wireless data exchange therewith.

A power supply module 166 is electrically connected to all modules in the meter chassis 108 and to the processing unit 172 to supply electric power thereto. A power supply line is also connected to chassis electrical interface 180, providing a chassis power interface, for supplying power to the analytical module 104 connected thereto whose module electrical interface 137 includes a corresponding power supply line and is configured to received electrical power therefrom and provide power to components of the analytical module 104 including, as necessary, electrical power to carry out analyte measurements. The power supply module 166 may comprise standard or rechargeable batteries 168 or an AC power supply 167 that may be activated when the analytical test meter 100 is connected to a source of AC power. The power supply module 166 is also electrically connected to processing unit 172 over the electrical interface 173 so that processing unit 172 can monitor a power level remaining in the battery 168.

As shown in FIG. 3, a functional schematic architecture is provided with regard to a modular analytical module 104, such as depicted in FIG. 2A. According to this exemplary version, the analytical plug-in module 104 and meter chassis 108 are each provided with various functionalities and with a mechanical and electrical interface therebetween, as described above. The meter chassis 108 and analytical plug-in module 104, when engaged, function as a complete analytical test meter 100. In terms of overall versatility and options as discussed herein, a user can be provided with a complete system that includes both a meter chassis 108 and analytical plug-in module 104, or a separate meter chassis 108 and/or at least one separate analytical plug-in module 104.

With reference to FIG. 3, in one embodiment a current measurement method and circuit is used to measure a selected analyte concentration using the sample analyte measurement engine, such as a blood glucose measurement engine (BG engine) 302. A microcontroller 320 is embedded in the analytical module 104 and controls operations of the analytical module 104. The microcontroller may be in the form of a MSP430V346 Mixed Signal Microcontroller made by Texas Instruments Corp. of Dallas, Tex. This microcontroller 320 includes a processor and system memory such as SRAM and flash for storing programs and data, ultralow power consumption, universal serial bus (USB), a 12-bit ADC, and signal generators sufficient for performing blood analyte measurement operations as described herein.

The BG engine 302 is in electrical communication with the SPC 139 and the microcontroller 320 to detect a resistance magnitude change across electrodes of a test strip 14 inserted into SPC 139, which indicates that a blood sample has been applied thereto, using a potentiostat. At a predetermined time after the blood sample has been applied to the test strip 14, a voltage signal is applied across the sample via the electrodes 13 which generates an electric current therethrough. The BG engine 302 converts the electric current measurement into digital form, such as an analyte concentration in standard units (e.g., mmol/L or mg/dL) for transmission across the electric interface 137 to the meter chassis 108 for presentation on the display 114. The processing unit 172 is configured to receive input in the form of digital data from the BG engine 302 via electrical interface 180.

The analyte test strip 14 can be in the form of an electrochemical glucose test strip defined by a nonporous substrate that can include one or more working electrodes 13. Test strip 14 can also include a plurality of electrical contact pads, where each electrode can be in electrical communication with at least one electrical contact pad. Strip port connector 139 can be configured to electrically interface to the electrical contact pads, using electrical contacts in the form of prongs, and form electrical communication with the electrodes. Test strip 14 can include a reagent layer that is disposed over one or more electrodes within the test strip 14, such as a working electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). An exemplary mediator suitable for use in the reagent layer includes ferricyanide, which in this case is in the oxidized form. The reagent layer can be configured to physically transform glucose in the applied sample into an enzymatic by-product and in the process generate an amount of reduced mediator (e.g., ferrocyanide) that is proportional to the glucose concentration of the sample. The working electrode can then be used to apply the voltage signal to the sample and to measure a concentration of the reduced mediator in the form of an electric current, and communicate measurement information in digital form to the microcontroller 172, which can present the information in a human readable form on the display 114. An exemplary analytical test meter performing such current measurements is described in U.S. Patent Application Publication No. US 2009/0301899 A1 entitled “System and Method for Measuring an Analyte in a Sample”, which is incorporated by reference herein as if fully set forth in this application.

In addition to the mechanical keying described above, electrical keying may further be provided, in addition to or as a replacement for the mechanical keying, as part of the overall interface between the analytical plug-in module 104 and the meter chassis 108 in order to ensure that the analytical module 104 is engaged with a correctly authorized meter chassis 108 or to ensure that a firmware update is authorized for use by the meter chassis 108. The firmware update may be stored in an EEPROM, ROM, or Flash memory provided with the analytical module 104 as a code image 306. An example of an authorized firmware update may include the addition of a software based bolus calculator for patients who also wear an insulin patch and desire to use the tool for tracking insulin use patterns and remaining insulin dosages. Those meter chassis that are unable to install and execute bolus calculator software would recognize such a software tool as unauthorized.

As an example of a firmware update, various enhanced strip platforms or tool versions may become available for installation on a variety of different meter chassis 108, in which updated, new feature capabilities may be enabled for particular meter chassis 108. Such new features may include an algorithm enabled by a newly developed strip technology or platform. Firmware upgrades may be directed to other aspects of meter operations. For example, newly developed diabetes management tools stored in the analytical module may be transmitted to the meter chassis 108. Some of these tools can be programmed to detect patterns in blood glucose measurements, and may be designated as premium tools to be made available for a nominal increase in price. Another example of a firmware update may include the addition of a software based bolus calculator, described above, for patients who wear an insulin patch.

In at least one version, the analytical plug-in module 104 may store one or more firmware updates thereon with corresponding firmware codes, such as a numerical identifier, corresponding to one or more different chassis types. Upon insertion into, and electrical engagement with, a meter chassis 108, a programmed protocol may be initiated in the meter chassis 108 or in the analytical module 104 whereby the firmware codes and meter chassis types, which may be another numerical identifier stored on the chassis side, are compared and verified so that only authorized updated platform or tool versions may be installed in the meter chassis 108. After the firmware codes are properly identified as compatible, a bootstrap loader 304 pushes the code image over the module electrical interface 137 which is stored and installed for use in the meter chassis 108 by the processing unit 172. Such a protocol may be programmed to be automatically initiated and performed upon insertion of an analytical module 104 into the meter chassis 108. The Bootstrap loader 304 may be required when the code image contains the complete firmware for the meter chassis 108, and therefore the old chassis firmware is erased before loading the new firmware. During that duration the meter chassis 108 does not participate in the code transfer. Alternatively, if new tools are loaded in addition to the existing meter chassis code, then a code image transfer mode may be implemented where the meter chassis 108 remains active and may control or otherwise participate in the code transfer.

Electronic data signal lines 308 are electrically connected to the module electrical interface 137 and to the components of the analytical module 104 just described. The signal lines may include, but are not limited to, power and ground lines 310, data signal transmit/receive lines 312, interrupt and reset signal lines 314, strip insertion and/or removal detection lines 316, and mode select lines 318. Operation of such signal lines are well known to those persons having ordinary skill in the art. Thus, circuit design considerations such as data transmission rates, voltage levels, handshaking protocols, serial/parallel transmission, and synchronous or asynchronous transmission, for example, are not considered to be significant with respect to the embodiments described herein. The mode select lines 318 may be used to select a functional mode of the analytical module 104 such as between a blood sample assay mode and a firmware transmission mode, for example.

According to at least one embodiment, and because the meter chassis 108 supplies power to the analytical module 104, the meter chassis 108 can detect when an analytical plug-in module 104 is either not connected to the meter chassis 108 or is improperly or incorrectly connected to the meter chassis 108, a state herein referred to as “auto empty detection”. When there is a failure by the microcontroller 172 of the meter chassis 108 to detect an analytical plug-in module or a properly fitted analytical module (either mechanically or electrically), the microcontroller may be programmed to remain in a low power or passive mode of operation. Alternatively, or in addition to the above, a warning indication may be provided to the user by either at least one visual (displayed) and/or audio signal. According to yet another alternative, an on-screen tutorial may be stored in meter chassis memory 151 and presented via the display 114 in order to provide user instructions for properly connecting an analytical plug-in module 104 to the meter chassis 108.

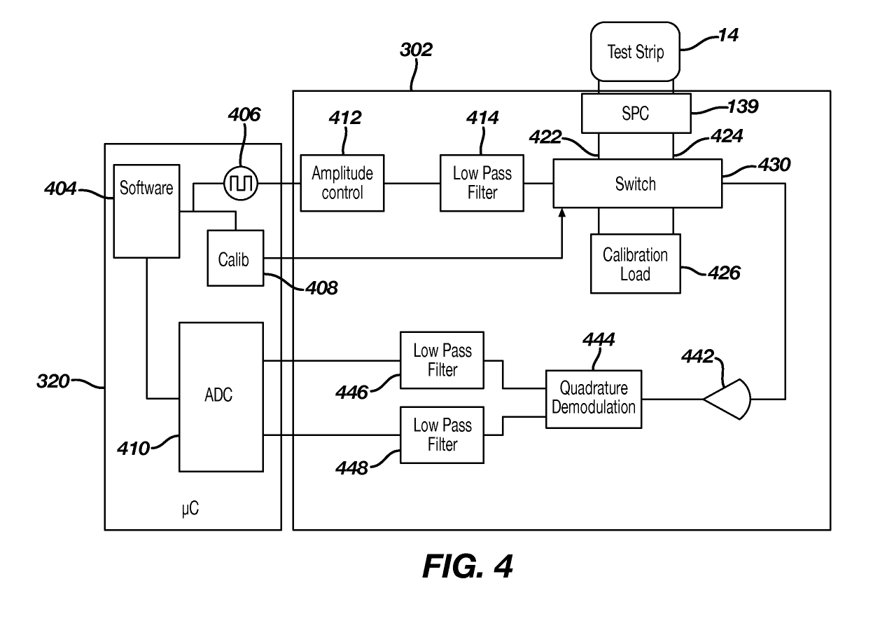

With reference to FIG. 4, in another embodiment a phase and magnitude measurement method and circuit is used to measure a selected analyte concentration, such as blood glucose, using the blood glucose measurement engine (BG engine) 302. As illustrated, the BG engine 302 is electrically connected to the strip port circuit 139 and to the microcontroller 320 as described above. Operation of the BG engine 302 is controlled by the microcontroller 320. In principle, the BG engine 302 drives a known electrical sine wave signal through the test strip 14 having a blood sample thereon in order to measure its effect on the magnitude and phase of the electrical sine wave signal applied thereto. The circuit 139 comprises at least two electrical contacts 422 and 424 connected to the electrodes of an inserted test strip 14 having a blood sample thereon. In operation, a square wave generator 406 transmits a square wave signal through an amplitude control block 412, which sets a precise amplitude of the square wave, and through a low pass filter 414 which converts the square wave to a sinusoidal wave. This sine wave input signal is driven through the test strip 14 strip via the electrical contact 422 in electrical communication with a test strip electrode. The electrical properties of the blood sample in the test strip 14 affect the magnitude and phase of the electrical sine wave input signal that passes through it. Depending on properties of the blood sample, e.g. analyte levels in the blood, such as hematocrit, the sample presents a corresponding impedance to the sine wave which, in turn, affects the phase and magnitude of the sine wave passing through it. The affected (modified) sine wave output from a test strip electrode to contact 424 is transmitted through a transimpedance amplifier 442 to condition the signal before it is fed through a quadrature demodulator 444. The quadrature demodulator 444 decomposes the sinusoidal voltage signal into measurable real and imaginary components. These components are each filtered by one of the low pass filters 446, 448 and are received at the ADC 410 in the microcontroller 320. The phase and magnitude of the modified waveforms are calculated by microcontroller 320 according to software programs 404 (as part of data stored in a memory of the microcontroller 320) based on the real and imaginary components of the received output signal and on calibration parameters generated during a calibration phase of the BG engine 302 (described below). Thus, the BG engine 302 drives a known sine wave through the test strip 14 having a blood sample on it to measure its magnitude and phase effects on the applied known sine wave.

During a calibration phase, performed after test strip insertion but before a sample is applied thereto by a user, a known calibration load 426 is switched into the BG engine 302 by electronic switch 430. Under direction from microcontroller 320, the switch 430 can controllably connect the contacts 422 and 424 to the calibration load 426, or to the test strip 14 for analyte level measurement. Prior to the actual test strip sample analyte measurement, microcontroller 320 selectively connects the contacts 422, 424 to the known calibration load 426 during hardware integrity checks, calibration of impedance circuits with respect to voltage offsets and leakage currents, and the like. The test strip is switched in for actual testing after calibration is completed, wherein the user applies a sample to the test strip for analyte measurement. Calibration parameters generated during this calibration phase are used to adjust the magnitude and phase calculations as described above.

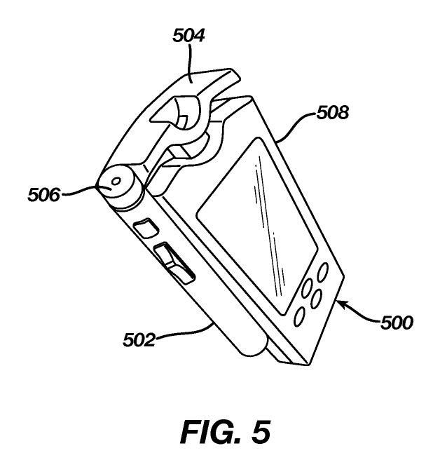

As shown in FIG. 5, another exemplary analytical test meter 500 is provided, the test meter 500 also having an analytical plug-in module 504 that is mechanically and electrically interconnected in a releasable fashion to a meter chassis 508. As in the preceding described version, the plug-in analytical module 504 is defined by a strip port connector and retains software that enables an analyte of interest to be measured in which the meter chassis 508 and analytical components are configured to cooperate with one another upon attachment. According to this exemplary version, another system component 502 such as a test strip dispenser, a strip ejector or lancing device can be combined with the plug-in analytical module 504 to provide additional versatility and capability. In the depicted version, a lancing device 506 is disposed on the analytical module 504 that can be or otherwise releasably or fixedly attached onto the side surface of the meter chassis 508. Alternatively, the lancing device 506, or other added system component can be provided as an integral part of the module 504. The added system component can be stored when the analytical plug-in module 504 is successfully engaged (keyed) with the meter chassis 508.

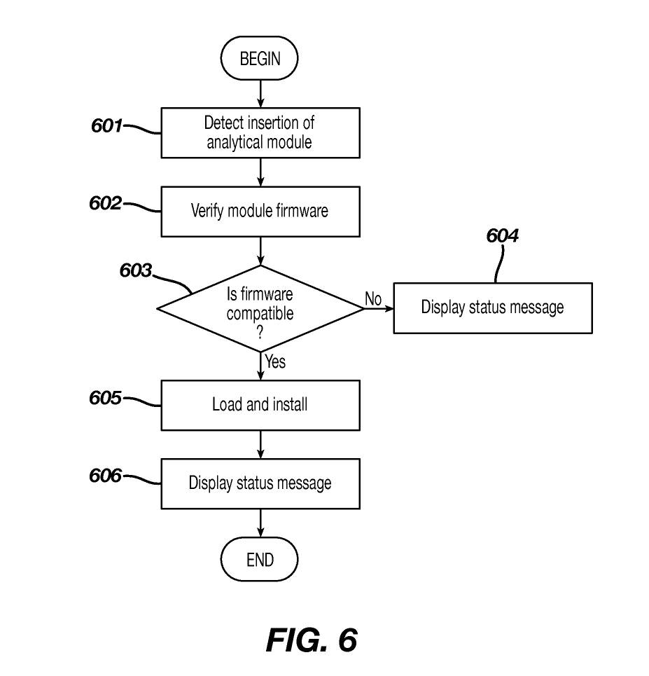

FIG. 6 illustrates an exemplary method of enabling a test meter in accordance with the present invention and more specifically with regard to analytical test meter 100. The method begins at step 601 with detecting an insertion of the analytical module 104 into meter chassis 108. As described above, the analytical module 104 receives power from the meter chassis 108 power supply module 166. The processing unit 172 may detect the insertion of the analytical module and begins a verification procedure, at step 602, whereby a firmware code or a module ID code stored in the analytical module is read and compared with a meter chassis ID stored in a memory of the meter chassis 108. If the inserted module's firmware code or module ID is determined by the processing unit 172 as not being an authorized and compatible module, at step 603, firmware is not transmitted over the mechanical and electrical interface and the analytical module 104 is not rendered operable with the test meter 100. An incompatible module may be detected if the module is an older version or if the firmware stored thereon is incompatible with the meter chassis hardware. A stored status message may then be displayed on a display screen 114 of the meter chassis 108, at step 604, to indicate to a user of the analytical test meter 100 that the analytical module 104 is not enabled. If the inserted module's firmware code or module ID is determined by the processing unit 172 as being an authorized and compatible module, at step 603, firmware corresponding to the test meter 100 type is transmitted over the mechanical and electrical interface using the module's bootstrap loader for installation by the processing unit 172 at step 605. A stored status message may then be displayed on a display screen 114 of the meter chassis 108, at step 606, to indicate to a user of the analytical test meter 100 that the test meter 100 has been updated with the new firmware.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “circuit,” “circuitry,” “module,” “subsystem” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible, non-transitory medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Furthermore, the various methods described herein can be used to generate software codes using off-the-shelf software development tools. The methods, however, may be transformed into other software languages depending on the requirements and the availability of new software languages for coding the methods.

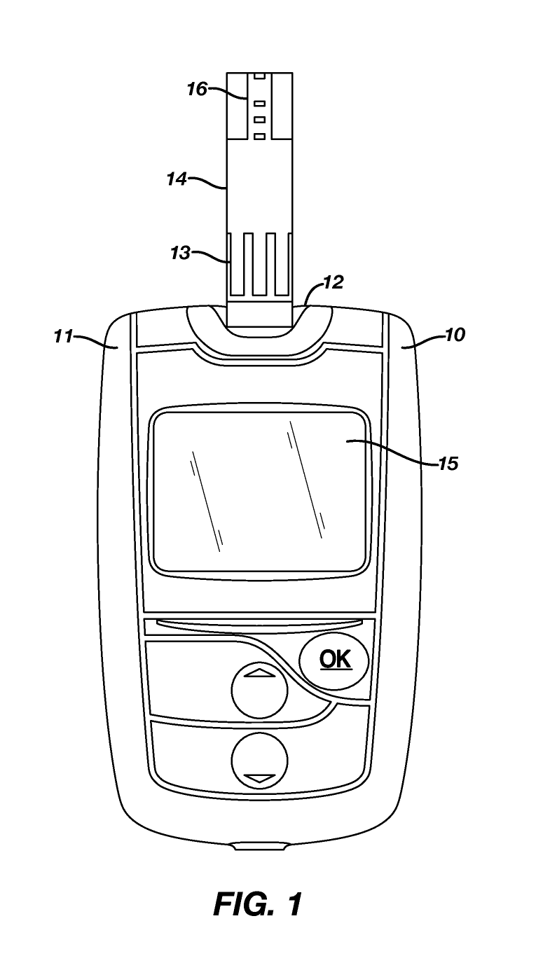

### PARTS LIST FOR FIGS. 1-6

10 test meter

11 housing, meter

12 strip port opening

13 test strip electrodes

14 test strip

15 display

16 electrochemical cell

100 analytical test meter

104 analytical plug-in module

108 meter chassis

112 module housing

113 distal end, module

114 display

115 proximal end, module

116 engagement pin

120 engagement pin

122 strip port opening

124 spaced opening

128 spaced opening

129 proximal end, chassis

132 user interface buttons

134 chassis body top surface

136 arrow

137 module electrical interface

138 enclosure

139 strip port connector

141 sized recess

144 chassis housing

150 data management unit

151 memory module

152 buttons module

153 user interface module

155 microcontroller settings module

156 transceiver module

157 antenna

158 WiFi module

159 Bluetooth module

160 NFC module

162 RAM module

163 ROM module

164 external storage

165 light source module

166 power supply module

167 AC power supply

168 battery power supply

169 display module

170 audio module

171 speaker

172 microcontroller (processing unit)

173 communication interface

180 chassis electrical interface

302 blood glucose engine

304 bootstrap loader

306 code image

308 interface signal lines

310 power and ground lines

312 data signal transmit/receive signal lines

314 interrupt and reset signal lines

316 strip insertion and removal detection signal lines

318 mode select signal lines

404 software

406 squarewave generator

408 calibration control

410 analog-to-digital converter (ADC)

412 amplitude control

414 low pass filter

422 test strip electrode

424 test strip electrode

426 calibration load

430 switch

442 transimpedance amplifier

444 quadrature demodulator

446 low pass filter

448 low pass filter

500 test meter

502 added system component

504 analytical module

506 lancing device

508 meter chassis

601 step—detect insertion of analytical module

602 step—verify module firmware

603 step—is firmware compatible

604 step—display status message

605 step—load and install

606 step—display status message

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. An analytical test meter for determining a concentration of an analyte of interest in a bodily fluid sample, the test meter comprising:
a meter chassis comprising a chassis electrical, mechanical, and power interface;
a plug-in analytical module comprising a module electrical, mechanical, and power interface that is mechanically manually connectable to, and disconnectable from, said meter chassis, said analytical module including:
a strip port connector configured for receiving an inserted test strip;
a mechanical and electrical module interface;
a measurement engine configured for detecting and measuring the concentration of the analyte of interest of the inserted test strip; and
a module microcontroller connected between the measurement engine and the mechanical and electrical module interface to electronically communicate the measured concentration of the analyte of interest over the electrical interface; and
wherein the electrical, mechanical and power interface of the plug-in analytical module is configured for operational engagement with the electrical, mechanical and power interface of the meter chassis in a removable and replaceable manner.

2. The test meter of claim 1, wherein said analytical module further comprises a memory for storing chassis firmware and said meter chassis comprises a chassis microcontroller and memory for executing the chassis firmware, the meter chassis and the analytical module configured to automatically transmit the chassis firmware from the analytical module to the meter chassis upon manually connecting the analytical module to the meter chassis.

3. The test meter of claim 2, wherein said chassis firmware comprises a chassis firmware update, an enhanced analyte management tool, an algorithm to utilize a newly developed test strip technology, a bolus calculator, a tool for detecting patterns in analyte measurements, or a combination thereof.

4. The test meter of claim 1, wherein said meter chassis includes a mechanical and electrical chassis interface for connecting to the module interface to transmit electronic data to the analytical module and to receive electronic data from the analytical module.

5. The test meter of claim 4, wherein said meter comprises a display screen to display the electronic data received over the mechanical and electrical chassis interface, the electronic data including the measured concentration of the analyte of interest.

6. The test meter of claim 1 in which the meter chassis and analytical module are interconnected to one another using at least one of a mechanically keyed arrangement and an electrically keyed arrangement.

7. The test meter of claim 6, wherein the meter chassis is configured to releasably receive a plurality of different analytical modules, each of said analytical modules configured to measure an analyte of interest.

8. The test meter of claim 1, in which the meter chassis includes a display and a microcontroller for controlling a user interface presented on the display when the analytical module is connected to the meter chassis.

9. The test meter of claim 1, wherein said analytical module includes volatile memory, non-volatile memory, or a combination thereof.

10. The test meter of claim 9, wherein said non-volatile memory includes an EEPROM to store firmware to be installed in the meter chassis upon attachment of said analytical module.

11. An analytical test meter comprising:
a meter chassis comprising a chassis interface; and
a first module comprising:
a first module interface that is releasably connectable to, and disconnectable from, the chassis interface;
a strip port connector for receiving a first test strip;
a resident sample analyte engine for performing a first assay on a sample received on the test strip; and
a resident circuit for electronically transmitting a result of the first assay to the meter chassis over the first module interface.

12. The analytical test meter of claim 11, further comprising:
a second module comprising a second module interface that is releasably connectable to, and disconnectable from, the chassis interface, the second module comprising a strip port connector for receiving a second test strip having at least one feature different from the first test strip and for performing a second assay on a second sample received on the second test strip, the second module including a circuit for electronically transmitting a result of the second assay to the meter chassis over the second module interface.

13. The analytical test meter of claim 11, wherein the first module is configured to perform the first assay only after the first module interface engages the chassis interface.

14. The analytical test meter of claim 11, wherein the first module is configured to perform the first assay only after both the first module interface engages the chassis interface and the first module transmits firmware stored by the module over the module interface to be installed in the meter chassis.

15. The analytical test meter of claim 11, wherein the chassis interface comprises a mechanical chassis interface and the first module interface comprises a mechanical module interface, wherein the mechanical chassis interface prevents connection of the mechanical module interface thereto if the first module is electrically incompatible with the meter chassis.

16. The analytical test meter of claim 11, wherein the chassis interface comprises an electrical chassis interface and the first module interface comprises an electrical module interface, wherein the electrical chassis interface prevents operation of the first module if the first module is electrically incompatible with the meter chassis.

17. A method for enabling a modular analytical test meter, said method comprising:
providing a meter chassis;
providing an analytical module having a strip port connector disposed therein configured for receiving an analytical test strip;
engaging said analytical module with said meter chassis, said analytical module and said meter chassis including complementary mating features, said analytical module being configured for measuring an analyte of interest from a test strip inserted into said strip port connector having a sample placed thereon and relaying the measured result to a processor of said meter chassis.

18. The method of claim 17, further comprising:
said analytical module applying a voltage signal to said test strip having the sample placed thereon; and
detecting a response to the voltage signal including calculating an analyte concentration of the sample based on the response to the voltage signal.

19. The method of claim 17, further comprising said processor reading a digital code stored in the analytical module and verifying that the analytical module is properly configured, said code including a numerical identifier.

20. The method of claim 19, further comprising:
said properly configured analytical module electronically transmitting firmware data to the processor;
the processor installing the firmware data; and
the firmware data enabling the meter chassis to interoperate with the properly configured analytical module.
